(19) United States
(12) Patent Application Publication
(10) Pub. No.: US 2007/0259820 A1
(43) Pub. Date: Nov. 8, 2007

(54) METHODS AND REAGENTS FOR ACTIVATING HEAT SHOCK PROTEIN 70

(75) Inventors: Jason E. Gestwicki, Ann Arbor, MI (US); Christopher G. Evans, Ann Arbor, MI (US); Susanne Wisen, Ann Arbor, MI (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE REGENTS OF THE UNIVERSITY OF MICHIGAN, Ann Arbor, MI (US)

(21) Appl. No.: 11/743,439

(22) Filed: May 2, 2007

Related U.S. Application Data
(60) Provisional application No. 60/797,478, filed on May 3, 2006.

(51) Int. Cl. A61K 38/05; C07K 5/08

(52) U.S. Cl. 514/18; 514/269; 530/331

(57) ABSTRACT

The present disclosure is directed to new compounds useful as modulators of Heat Shock Proteins (HSP). In particular, the present disclosure provides new small molecule peptide-derived compounds having HSP modulation activity, especially Hsp70 modulation activity, and methods of preventing or ameliorating beta-amyloid or polyglutamine aggregation, decreasing cell proliferation, or increasing chaperon activity of the HSPs.

### CROSS-REFERENCE TO RELATED APPLICATIONS

This application claims the benefit of U.S. Provisional Application No. 60/797,478, filed May 3, 2006, which is incorporated by reference in its entirety.

### BACKGROUND

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation, and the majority of malignant cancers are facilitated by multiple genetic abnormalities in these pathway. Therefore, it is unlikely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation, and oxygen deprivation. HSPs act as chaperones to other cellular proteins (called client proteins) and facilitate their proper folding and repair, and aid in the refolding of misfolded client proteins. There are several known families of HSPs, each having its own set of client proteins. The Hsp70 family is one of the HSP families.

Hsp70 has three domains: a nucleotide-binding domain (NBD) with weak ATPase activity, a substrate-binding domain (SBD) and a C-terminal helix “lid” region. Via its SBD, the chaperone broadly recognizes exposed hydrophobic regions; these normally buried sequences are characteristic of misfolded peptides. The ADP-bound form of Hsp70 has a 10-50-fold better affinity for substrates than the ATP-bound form. Because of this difference, cycles of nucleotide hydrolysis cause iterative binding and release of substrates. These cycles minimize aggregation by sterically precluding non-productive interactions with the hydrophobic patches. Nucleotide turnover is regulated by co-chaperones; Hsp40 (or DnaJ) accelerates ATP hydrolysis and nucleotide exchange factors (NEFs), such as BAG-1 and GrpE, promote ADP release.

Hsp70 has also been reported to cooperate with Hsp90 as part of a complex as a chaperone. Therefore, the ability to block both Hsp70 and Hsp90 can be beneficial.

Surprisingly, there are very few chemical compounds that are known to interact with any of the molecular chaperones. The best studied is the Hsp90 inhibitor and natural product geldanamycin; derivatives of this compound are in clinical trials for anti-cancer therapy. In the 1980s, the natural product, spergaulin, was discovered and found to bind Hsp70 in “pull-down” studies. Subsequent efforts to find analogs provided R/1 and 15-deoxyspergualin (DSG), which were found to stimulate Hsp70's ATPase activity. Another recently reported molecule which interacts with HSP is MAL3-101.

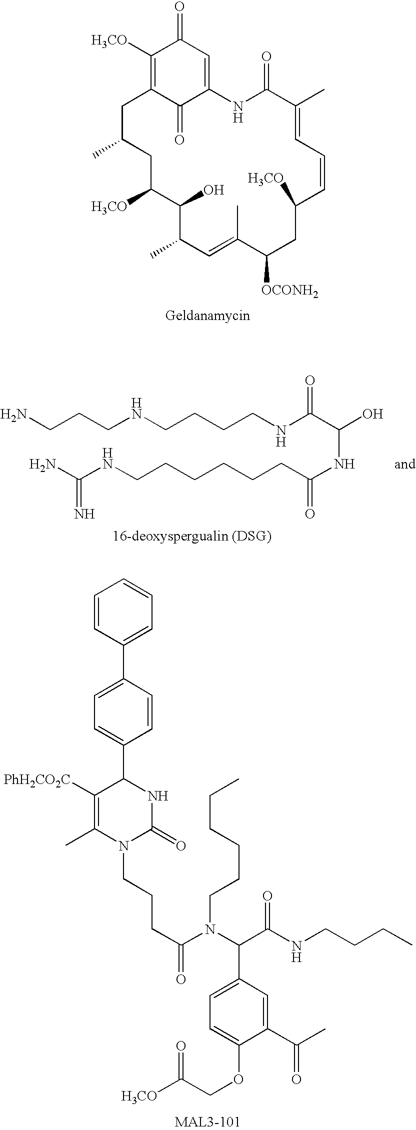

All of these compounds have been shown to have some effect on HSP activity, but few compounds have been identified as modulators of Hsp70 activity. Thus, there exists a need in the art to identify compounds that modulate Hsp70 activity.

### SUMMARY OF THE INVENTION

The present invention is directed to identification and use of compounds that modulate Hsp70 activity. For example, the invention provides small molecules and compositions as well as therapeutic compositions and uses of specific small molecule compounds.

Compounds suitable for use with the disclosed methods and compositions include those having a formula as described below, specifically formula (I): 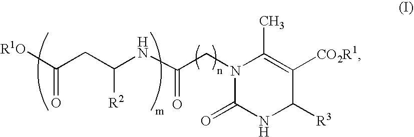
wherein R¹ is independently selected from the group consisting of C₁₋₈ alkyl and H; R² is independently selected from the group consisting of H, C₁₋₈alkyl, C₁₋₈alkylenethiol, C₁₋₈alkylenehydroxy, C₁₋₈alkyleneCO₂H, C₁₋₈alkyleneCO₂C₁₋₈alkyl, C₁₋₈alkyleneC(O)NHC₁₋₈alkyl, aryl, substituted aryl, CH₂aryl, CH₂substituted aryl, CH₂heteroaryl, and CH₂substituted heteroaryl; R³ is selected from the group consisting of aryl, substituted aryl, heteroaryl, and substituted heteroaryl; m is an integer selected from the group consisting of 0, 1, 2, 3, and 4; and n is an integer selected from the group consisting of 1, 2, 3, 4, 5, 6, and 7. In some embodiments, R³ is aryl or substituted aryl. In specific embodiments, R³ is selected from the group consisting of biphenyl, 2-thiophene, 2-hydroxy-1-naphthyl, 4-bromophenyl, 4-nitrophenyl, and 2-chlorophenyl. In various embodiments, R¹ is hydrogen, methyl or ethyl.

In specific embodiments, the compounds of formula (I) include:

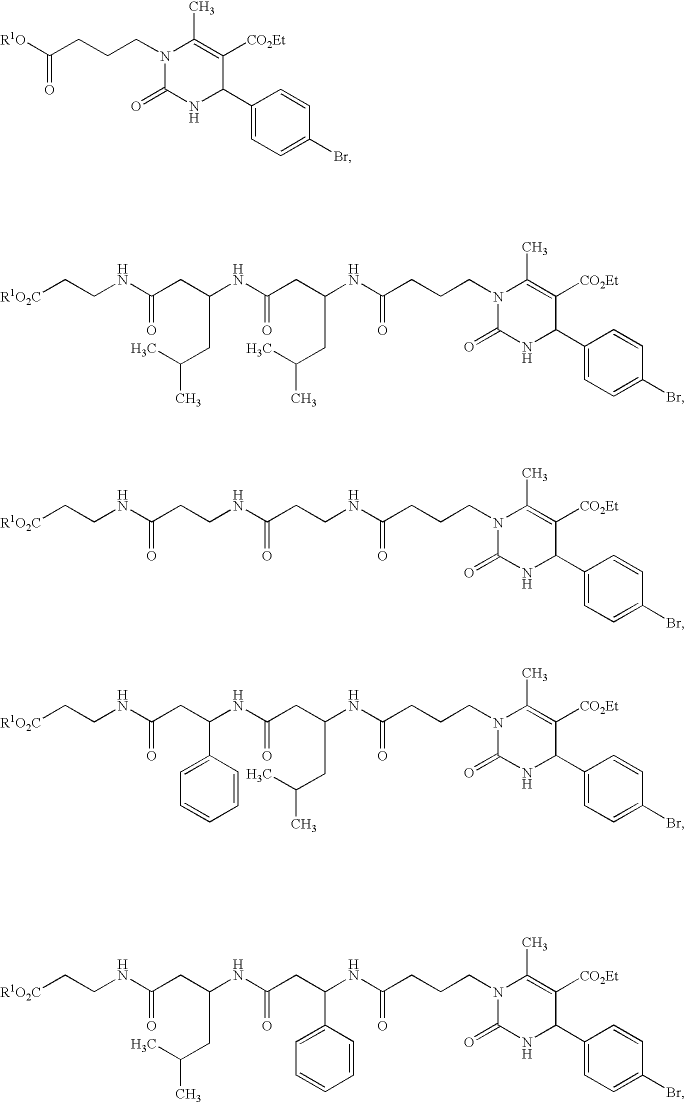

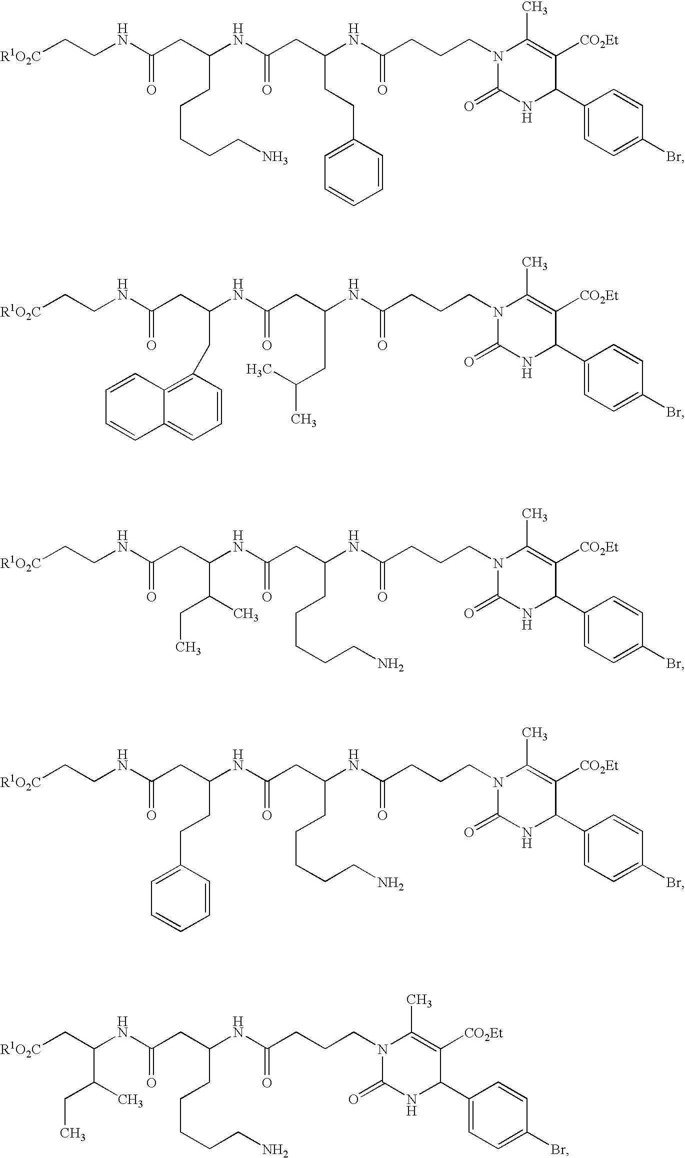

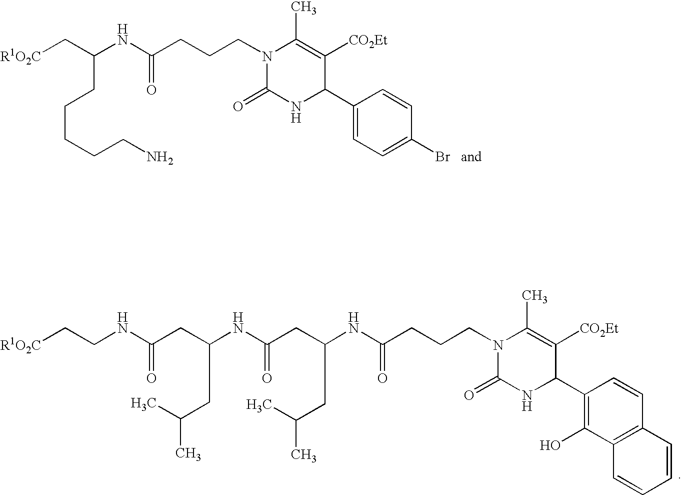

In one variation, the molecules themselves are the invention, preferably in a purified and/or isolated form. In another variation, the invention is a composition comprising one or more molecules of the invention—preferably purified and/or isolated—in admixture with a pharmaceutically acceptable diluent, adjuvant, excipient, or carrier. In another variation, the invention is a unit dosage formulation comprising a therapeutically effective amount of a molecule of the invention. In yet another variation, the invention is a sustained release formulation comprising a purified molecule of the invention.

Throughout this document, references to “compound” or “compounds” of the invention should be understood to refer to the compounds themselves, and also pharmaceutically acceptable salts, esters, prodrugs, and other formulations suitable for in vitro, in vivo, and ex vivo delivery of the active moiety to target cells.

The present invention also includes therapeutic methods comprising use of the compounds disclosed herein. An exemplary method of treatment comprises selecting a patient in need of treatment for a particular disorder, and administering to the patient an amount of a compound or composition of the invention effective to treat that disorder. The selecting step of the patient involves identifying the disorder by a review of a patient's medical records, a physical examination, a diagnostic test or interpretation of such test performed on the patient or on a biological sample (tissue, fluid, etc.) from the patient, or the like. The administering step can be by any route of administration, many of which are described herein. In specific embodiments, the compounds are used in treating patients suffering from aberrant cell proliferative disorders, beta-amyloid protein aggregation, and/or polyglutamine protein aggregation. Proliferative disorders include, but are not limited to, malignant gliomas, breast cancer, basal cell carcinoma, medulloblastomas, neuroectodermal tumors, and ependymomas. Beta-amyloid protein aggregation is typically associated with Alzheimer's disease. Polyglutamine protein aggregation is typically associated with Huntington's disease.

In addition to the foregoing, the invention includes, as an additional aspect, all embodiments of the invention narrower in scope in any way than the variations specifically mentioned above. For example, to the extent aspects of the invention have been described using ranges or genera for the sake of brevity, it should be understood that every sub-range, every individual value within a range, every subgenus, and every species are individually contemplated as a separate aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others.

### BRIEF DESCRIPTION OF THE DRAWINGS

FIG. 1 shows various compounds of formula (I) and their activity in refolding luciferase in the present of rabbit reticulocyte lysate (a source of Hsp70).

FIG. 2 shows (a) MCF7 cell viability for the 16 compounds presented in FIG. 1 and (b) sensitization of cancer cells in the presence and absence of compound 4 and/or geldanamycin.

FIG. 3 shows aggregation of beta-amyloid proteins in the presence and absence of compound 2, at varying concentrations (0.01, 0.1, and 0.5 mM).

FIG. 4 shows TEM analysis of amyloid structures after the indicated treatments for 50 minutes of 0.5 mM compound 2. Dark arrows indicate fibrils and light arrows point to other structures.

FIG. 5 shows thioflavin T fluoresence of samples after turbidity measurements (50 min) in the presence or absence of varying concentrations of compound 2. The results are the average of at least 3 experiments done in triplicate and the errors are standard deviations. The results are normalized to the buffer control (fluorescence between 20-50).

FIG. 6 shows effects of pharmacological Hsp70 modulation on polyQ aggregation in yeast. Yeast were transformed with a Q103 Htt-GFP construct. Individual colonies were removed from solid agar selection plates and placed into liquid YPD media. These suspensions were treated for 16 hours with compound 2. Between 200-400 yeast were imaged at 40× and scored as either aggregated or diffusely fluorescent. Sample images are shown.

### DETAILED DESCRIPTION OF THE INVENTION

Disclosed herein are new compound useful as modulators of Hsp70, specifically β-amino acid modified dihydropyrimidines of formula (I): 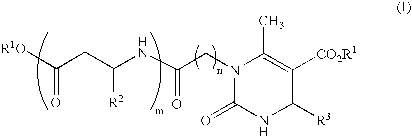
wherein R¹ is independent selected from the group consisting of C₁₋₈ alkyl and H; R² is independently selected from the group consisting of H, C₁₋₈alkyl, C₁₋₈alkylenethiol, C₁₋₈alkylenehydroxy, C₁₋₈alkyleneCO₂H, C₁₋₈alkyleneCO₂C₁₋₈alkyl, C₁₋₈alkyleneC(O)NHC₁₋₈alkyl, aryl, substituted aryl, CH₂aryl, CH₂substituted aryl, CH₂heteroaryl, and CH₂substituted heteroaryl; R³ is selected from the group consisting of aryl, substituted aryl, heteroaryl, and substituted heteroaryl; m is an integer selected from the group consisting of 0, 1, 2, 3, and 4; and n is an integer selected from the group consisting of 1, 2, 3, 4, 5, 6, and 7.

These compounds can be tailored using well known synthetic techniques to provide a wide variety of functionality and moieties to various parts of the molecules. For instance, β-amino acids can be employed having various side chains which can introduce hydrophobic, hydrophilic, bulky, etc. groups as desired to specific positions on the compounds of formula (I).

As used herein, the term “alkyl” refers to straight chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, ester, carboxylic acid, amide, guanidine, and amino.

As used herein, the term “alkylene” refers to an alkyl group having a substituent. For example, the term “alkenylene thiol” refers to an alkyl group substituted with a thiol (SH) group. The alkylene group is optionally substituted with one or more substituent previously listed as an optional alkyl substituent.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

As used herein, the term “protecting group” refers to a chemical group that exhibits the following characteristics: (1) reacts selectively with the desired functionality in good yield to give a protected substrate that is stable to the projected reactions for which protection is desired; (2) is selectively removable from the protected substrate to yield the desired functionality; and (3) is removable in good yield by reagents compatible with the other functional group(s) generated in such protection reactions. Examples of protecting groups can be found in Greene et al., “Protective Groups in Organic Synthesis,” 2d Ed. (John Wiley & Sons, Inc., New York, 1991).

Specific compounds contemplated include the following: 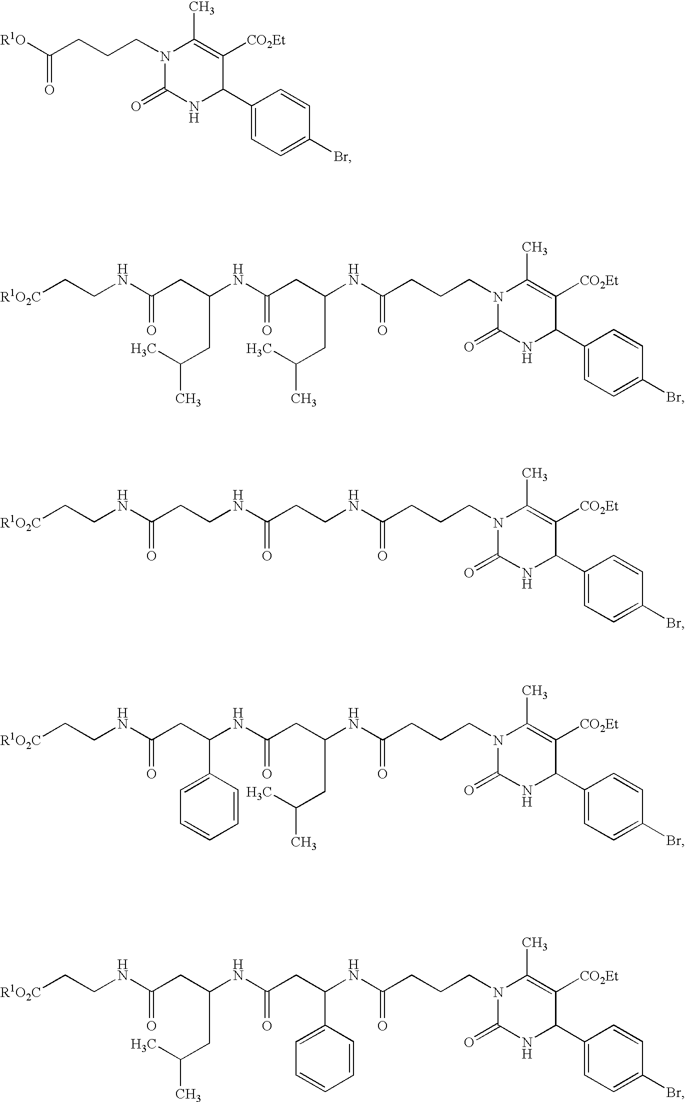 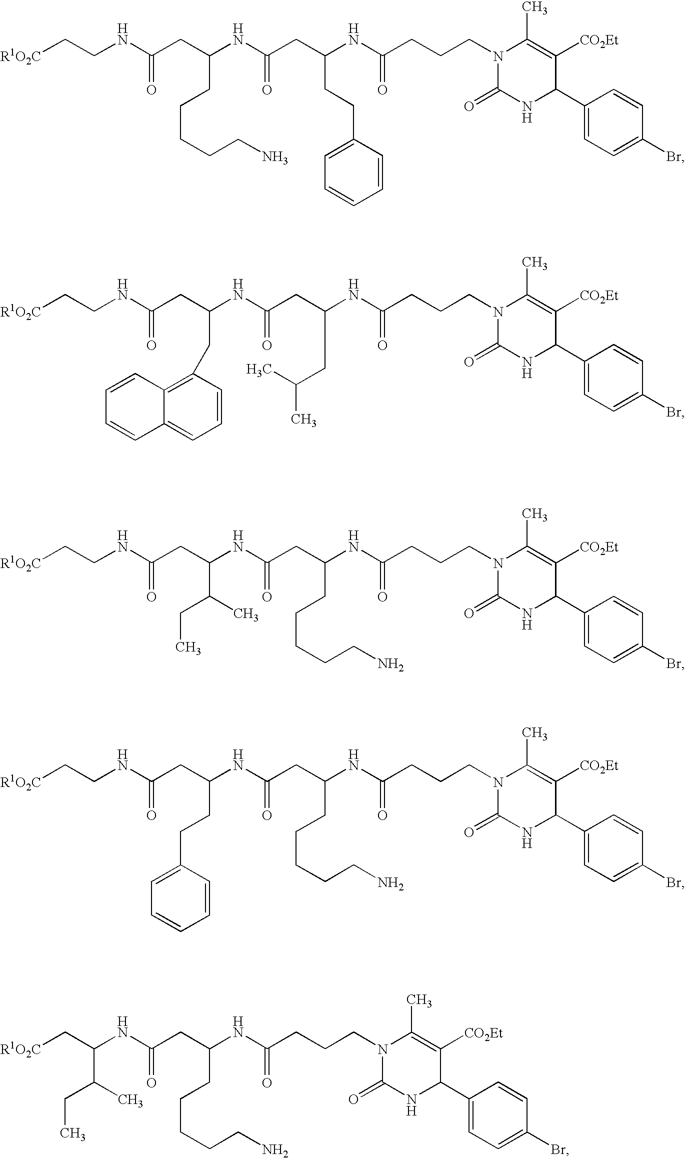 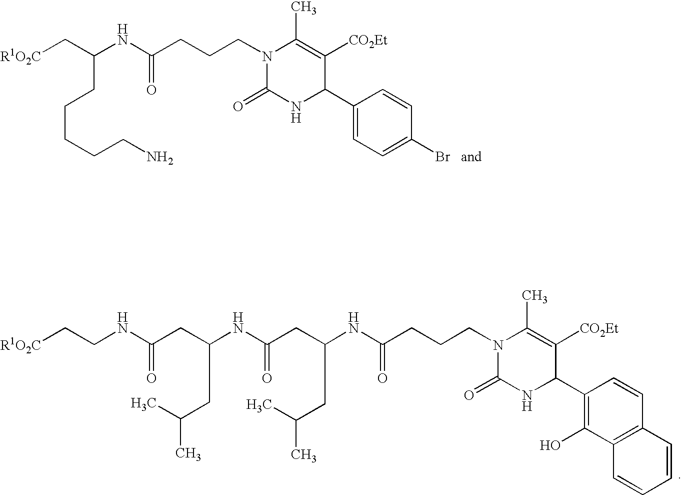
Synthesis of Compounds as Disclosed Herein

The compound disclosed herein can be prepared using a variety of methods available to the person of skill in the synthetic arts. Disclosed herein is one means for the synthesis which utilizes solid phase chemistry techniques to prepare the β-amino acid modified dihydropyrimidines. Scheme 1 outlines one such synthetic route. A solid phase resin is modified with one, two, three, four, or five β-amino acids in an iterative fashion to provide a small β-amino peptide. Next, a urea derivative of aminobutyric acid is coupled with the peptide. Next, a Biginelli reaction between the urea moiety, an aromatic aldehyde (R³CHO), and a keto-ester (CH₃C(O)CH₂CO₂R¹) is performed on the resin to prepare the dihydropyrimidine functionality. Last, the β-amino acid peptide modified dihydropyrimidine is cleaved from the resin to provide the compounds of interest.

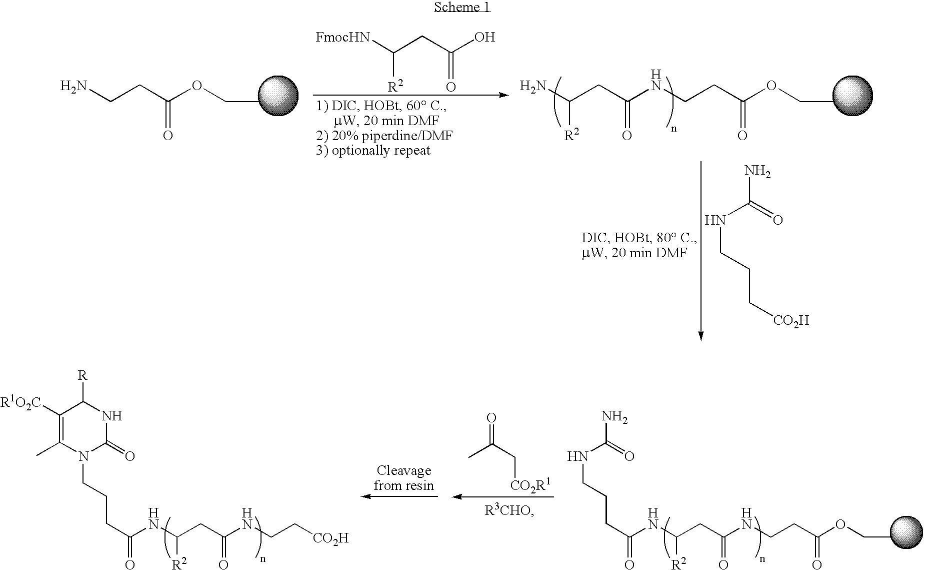

Any peptide coupling conditions can be used, including admixing the reagents (e.g., amine protected β-amino acids and free amine of the resin or previously coupled β-amino acid) in the presence of a coupling reagent. Coupling reagents include carbodiimides (e.g., DIC or DCC), HOBt, HOAt, HBTU, HATU, PyBOP, and the like. The amine protecting group can be any group stable to the coupling conditions, including, but not limited to, Fmoc, Boc, and benzyloxy carbonyl (Z or Cbz).

The compounds disclosed herein can exist as their corresponding salt, ester, or prodrug. As used herein, the term “pharmaceutically acceptable salts” refers to salts or zwitterionic forms of the compounds disclosed herein. Salts of such compounds can be prepared during the final isolation and purification of the compounds or separately by reacting the compound with an acid having a suitable cation. Suitable pharmaceutically acceptable cations include alkali metal (e.g., sodium or potassium) and alkaline earth metal (e.g., calcium or magnesium) cations. In addition, the pharmaceutically acceptable salts of the disclosed compounds that contain a basic center are acid addition salts formed with pharmaceutically acceptable acids. Examples of acids which can be employed to form pharmaceutically acceptable salts include inorganic acids such as hydrochloric, hydrobromic, sulfuric, and phosphoric, and organic acids such as oxalic, maleic, succinic, malonic, and citric. Nonlimiting examples of salts of compounds of the invention include, but are not limited to, hydrochloride, hydrobromide, hydroiodide, sulfate, bisulfate, 2-hydroxyethansulfonate, phosphate, hydrogen phosphate, acetate, adipate, alginate, aspartate, benzoate, butyrate, camphorate, camphorsulfonate, citrate, digluconate, glycerolphosphate, hemisulfate, heptanoate, hexanoate, formate, succinate, malonate, fumarate, maleate, methanesulfonate, mesitylenesulfonate, naphthylenesulfonate, nicotinate, oxalate, pamoate, pectinate, persulfate, 3-phenylpropionate, picrate, pivalate, propionate, trichloroacetate, trifluoroacetate, glutamate, bicarbonate, undecanoate, lactate, citrate, tartrate, gluconate, benzene sulphonate, and p-toluenesulphonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds appearing herein is intended to include compounds disclosed herein as well as pharmaceutically acceptable salts, solvates (e.g., hydrates), esters, or prodrugs thereof.

The compounds described herein and employed in the uses and methods of the present invention can exist in prodrug form. As used herein, the term “prodrug” is intended to include any covalently bonded carriers which release the active parent drug or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.), the compounds employed in the present methods can, if desired, be delivered in prodrug form. Thus, the present invention contemplates methods of delivering prodrugs. Prodrugs of the compounds employed in the present invention can be prepared by modifying functional groups present in the compound in such a way that the modifications are cleaved, either in routine manipulation or in vivo, to the parent compound.

Accordingly, prodrugs include, for example, compounds described herein in which a hydroxy, thiol, amino, or carboxy group is bonded to any group that, when the prodrug is administered to a mammalian subject, cleaves to form a free hydroxyl, thiol, free amino, or carboxylic acid, respectively. Examples include, but are not limited to, acetoxyalkyls, acetate, formate and benzoate derivatives of alcohol, thiol, and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

Compositions and Pharmaceutical Preparations of the Disclosed Compounds

Disclosed herein are compositions comprising the compounds as described above. The compositions comprise a therapeutically effective amount of the compounds or pharmaceutically acceptable salts thereof and a pharmaceutically acceptable carrier, adjuvant, and/or diluent.

The compounds are employed in amounts effective to achieve their intended purpose. As used herein, a “therapeutically effective amount” means an amount effective to inhibit development of, or to alleviate the existing symptoms of, the condition of the subject being treated. “Dose-effective to inhibit” means an amount effective to inhibit the aggregation of beta-amyloid proteins or polyglutamine proteins or decrease aberrant cell proliferation, in vitro, in vivo, or ex vivo. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio of LD₅₀ to ED₅₀. Compounds that exhibit high therapeutic indices (i.e., a toxic dose that is substantially higher than the effective dose) are preferred.

Modulation of Hsp70 can measured using a dose-response assay in which a sensitive assay system is contacted with a compound of interest over a range of concentrations, including concentrations at which no or minimal effect is observed, through higher concentrations at which partial effect is observed, to saturating concentrations at which a maximum effect is observed. Theoretically, such assays of the dose-response effect of compounds can be described as a sigmoidal curve expressing a degree of modulation as a function of concentration. The curve also theoretically passes through a point at which the concentration is sufficient to modulate activity of Hsp70 to a level that is 50% that of the difference between minimal and maximal activity in the assay. This concentration is defined as the Inhibitory Concentration (50%) or IC₅₀ value. Determination of IC₅₀ values preferably is made using conventional biochemical (acellular) assay techniques or cell based assay techniques.

Comparisons of the efficacy of compounds often are provided with reference to comparative IC₅₀ values, wherein a higher IC₅₀ indicates that the test compound is less potent, and a lower IC₅₀ indicates that the compound is more potent, than a reference compound. Compounds demonstrating IC₅₀ values of less than about 1500 μM, or less than about 1000 μM, or less than about 250 μM, or less than about 100 μM, or less than about 50 μM, or less than about 20 μM, or less than about 1 μM can be employed in compositions or methods according to the invention.

The data obtained in such dose-response assays can be used as a factor in formulating a dosage range for use in subject, such as animals, mammals, and more specifically, humans. The dosage of such compounds preferably lies within a range of circulating concentrations that include the ED₅₀ with little or no toxicity. The dosage can vary within this range depending upon the dosage form, and the route of administration utilized.

The exact formulation, route of administration, and dosage is chosen by a subject's physician, or treating professional, in view of the subject's condition. Dosage amount and interval can be adjusted individually to provide plasma levels of the active compound that are sufficient to maintain desired therapeutic effects. In general, however, doses employed for humans typically are in the range of 0.001 mg/kg to about 1000 mg/kg per day. In some embodiments, doses range from about 0.1 to about 50 mg/kg, about 0.5 to about 40 mg/kg, about 0.7 to about 30 mg/kg, or about 1 to about 20 mg/kg. Specific doses contemplated include sub-ranges of any of the foregoing ranges in 0.1 mg/kg increments.

The pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates,phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose, or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides (preferably sodium or potassium chloride); delivery vehicles; diluents; excipients and/or pharmaceutical adjuvants. (Remington's Pharmaceutical Sciences, 18th Edition, A. R. Gennaro, ed., Mack Publishing Company (1990).

The optimal pharmaceutical composition will be determined by one skilled in the art depending upon, for example, the intended route of administration, delivery format, and desired dosage. See, for example, Remington's Pharmaceutical Sciences, supra. Such compositions can influence the physical state, stability, rate of in vivo release, and rate of in vivo clearance of the compound of formula (I).

The primary vehicle or carrier in a pharmaceutical composition can be either aqueous or non-aqueous in nature. For example, a suitable vehicle or carrier can be water for injection, physiological saline solution, artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute therefore. The formulation components are present in concentrations that are acceptable to the route of administration. For example, buffers are used to maintain the composition at physiological pH or at a slightly lower pH, typically within a pH range of from about 5 to about 8.

The pharmaceutical compositions can be in the form of an aqueous, oleaginous suspension, dispersions or sterile powders, which can be used for the extemporaneous preparation of injectable solutions or dispersions. The suspension can be formulated according to the known art using those suitable dispersing or wetting agents and suspending agents which have been mentioned above. The compositions can also be solution or suspension in a non-toxic diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, fixed oils can be employed as a solvent or suspending medium. For this purpose, any bland fixed oil can be employed including synthetic mono- or diglycerides. In addition, fatty acids such as oleic acid find use in the preparation of injectables.

The disclosed compounds can be administered parenterally. Parenteral administration in this respect includes administration by the following routes: intravenous, intramuscular, subcutaneous, rectal, intraocular, intrasynovial, transepithelial including transdermal, ophthalmic, sublingual and buccal; topically including ophthalmic, dermal, ocular, rectal, and nasal inhalation via insufflation aerosol. When parenteral administration is contemplated, the therapeutic compositions for use in this invention can be in the form of a pyrogen-free, parenterally-acceptable aqueous solution comprising the Hsp70 modulator in a pharmaceutically acceptable vehicle. A particularly suitable vehicle for parenteral injection is sterile distilled water in which a Hsp70 modulator is formulated as a sterile, isotonic solution, properly preserved. Yet another preparation can involve the formulation of the desired molecule with an agent, such as injectable microspheres, bio-erodible particles, polymeric compounds (such as polylactic or polyglycolic acid), or beads or liposomes, that provide for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a Hsp70 modulator can be formulated as a dry powder for inhalation. Inhalation solutions can also be formulated with a propellant for aerosol delivery. In yet another embodiment, solutions can be nebulized. Pulmonary administration is further described in PCT application no. PCT/US94/001875, which describes pulmonary delivery of chemically modified proteins, but which can be applicable to pulmonary delivery of compounds as disclosed herein.

It is also contemplated that certain formulations can be administered orally. In one embodiment of the present invention, Hsp70 modulators which are administered in this fashion can be formulated with or without those carriers customarily used in the compounding of solid dosage forms such as tablets and capsules. For example, a capsule can be designed to release the active portion of the formulation at a point in the gastrointestinal tract when bioavailability is maximized and pre-systemic degradation is minimized. Additional agents can be included to facilitate absorption of the Hsp70 modulator. Diluents, flavorings, low melting point waxes, vegetable oils, lubricants, suspending agents, tablet disintegrating agents, and binders can also be employed.

Pharmaceutically acceptable ingredients are well known for the various types of formulation and can be for example binders such as natural or synthetic polymers, excipients, lubricants, surfactants, sweetening and flavouring agents, coating materials, preservatives, dyes, thickeners, adjuvants, antimicrobial agents, antioxidants and carriers for the various formulation types. Nonlimiting examples of binders useful in a composition described herein include gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers can be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, poly-acrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, und natural polymers like chitosan.

Nonlimiting examples of excipients useful in a composition described herein include phosphates such as dicalcium phosphate. Nonlimiting examples of lubricants use in a composition described herein include natural or synthetic oils, fats, waxes, or fatty acid salts such as magnesium stearate.

Surfactants for use in a composition described herein can be anionic, anionic, amphoteric or neutral. Nonlimiting examples of surfactants useful in a composition described herein include lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Nonlimiting examples of sweetening agents useful in a composition described herein include sucrose, fructose, lactose or aspartame. Nonlimiting examples of flavoring agents for use in a composition described herein include peppermint, oil of wintergreen or fruit flavors such as cherry or orange flavor. Nonlimiting examples of coating materials for use in a composition described herein include gelatin, wax, shellac, sugar or other biological degradable polymers. Nonlimiting examples of preservatives for use in a composition described herein include methyl or propylparabens, sorbic acid, chlorobutanol, phenol and thimerosal.

Another pharmaceutical composition can involve an effective quantity of Hsp70 modulator in a mixture with non-toxic excipients which are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving Hsp70 modulators in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. Additional examples of sustained-sustained-release preparations include semipermeable polymer matrices in the form of shaped articles, e.g. films or microcapsules. Sustained release matrices can include polyesters, hydrogels, polylactides (U.S. Pat. No. 3,773,919 and EP 058 481), copolymers of glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D-3-hydroxybutyric acid (EP 133 988). Sustained-release compositions also can include liposomes, which can be prepared by any of several methods known in the art. See e.g., Eppstein et al., Proc. Natl. Acad. Sci. USA, 82:3688-3692 (1985); EP 88 046; 036 676; and EP 143,949.

The pharmaceutical composition to be used for in vivo administration typically must be sterile. This can be accomplished by filtration through sterile filtration membranes. Where the composition is lyophilized, sterilization using these methods can be conducted either prior to, or following lyophilization and reconstitution. Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration. In addition, compositions can be placed into a container having a sterile access port, for example, an intravenous solution bag or vial having a stopper pierceable by a hypodermic injection needle.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

The compounds employed in the methods of the present invention can be administered by any means that results in the contact of the active agent with the agent's site of action in the body of a patient. The compounds can be administered by any conventional means available for use in conjunction with pharmaceuticals, either as individual therapeutic agents or in a combination of therapeutic agents. For example, they can be administered as the sole active agent in a pharmaceutical composition, or they can be used in combination with a second or additional therapeutic agent.

In specific embodiments, the compounds of this invention can, when used in cancer therapy, be used together with other substances and compounds, such as chemotherapeutic agents. Such compounds are, for example (according to the general classes of the compounds): Alkylating agents: Nitrogen mustards (mechlorethamine; cyclophosphamide; ifosfamide; melphalan; chlorambucil); Nitrosoureas (carmustine (BCNU); lomustine (CCNU); semustine (methyl-CCNU)); Ethylenimine/Methylmelamine (thriethylenemelamine (TEM); triethylene thiophosphoramide (thiotepa); hexamethylmelamine (HMM, altretamine)); Alkyl sulfonates (busulfan); Triazines (dacarbazine (DTIC)); and Antimetabolites (Folic Acid analogs—methotrexate and trimetrexate; Pyrimidine analogs—5-fluorouracil, fluorodeoxyuridine, gemcitabine, cytosine arabinoside, 5-azacytidine, 2,2′-difluorodeoxycytidine); Purine analogs—6-mercaptopurine, 6-thioguanine, azathioprine, 2′-deoxycoformycin (pentostatin), erythrohydroxynonyladenine (EHNA), fludarabine phosphate, and 2-Chlorodeoxyadenosine (cladribine, 2-CdA)); Type I Topoisomerase Inhibitors: camptothecin; topotecan; irinotecan; Natural products: Antimitotic drugs (paclitaxel; Vinca alkaloids—vinblastine (VLB), vincristine, and vinorelbine; Taxotere® (docetaxel); estramustine; estramustine phosphate); Epipodophylotoxins (etoposide and teniposide); Antibiotics (actimomycin D; daunomycin (rubidomycin); doxorubicin (adriamycin); mitoxantrone; idarubicin; bleomycins; plicamycin (mithramycin); mitomycinC; and dactinomycin); Enzymes (L-asparaginase); Biological response modifiers: interferon-alpha; IL-2; G-CSF; and GM-CSF; Differentiation Agents: retinoic acid derivatives; Radiosensitizers: metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine, Miscellaneous agents: Platinium coordination complexes (cisplatin, carboplatin); Anthracenedione (mitoxantrone); Substituted urea (hydroxyurea); Methylhydrazine derivatives (N-methylhydrazine (MIH) and procarbazine); Adrenocortical suppressant (mitotane (o,p′-DDD) and aminoglutethimide); Cytokines (interferon (α, β, γ) and interleukin-2); Hormones and antagonists: Adrenocorticosteroids/antagonists (prednisone and equivalents; dexamethasone; aminoglutethimide); Progestins (hydroxyprogesterone caproate; medroxyprogesterone acetate; megestrol acetate); Estrogens (diethylstilbestrol, ethynyl estradiol/equivalents); Antiestrogen (tamoxifen); Androgens (testosterone propionate, fluoxymesterone/equivalents); Antiandrogens (flutamide; gonadotropin-releasing hormone analogs, leuprolide); Nonsteroidal antiandrogens (flutamide); Photosensitizers: hematoporphyrin derivatives, Photofrin®, benzoporphyrin derivatives, Npe6, tin etioporphyrin (SnET2), pheoboride-a, bacteriochlorophyll-a, naphthalocyanines, phthalocyanines, and zinc phthalocyanines; Proteosome inhibitors: bortezomib (Velcade®). In addition to the above, there are several novel compounds disclosed in various patent applications that are contemplated as second therapeutic agents, e.g.: Epothilones (US 2005244413), serratamolide (US 2005239694), indol derivatives (US 2005239752), various plant extracts: extract of sea buckthorn—Hippophae rhamnoides (US 2005214394), extracts of Ganoderma lucidum, Salvia miltiorrhiza and Scutellaria barbata (US 2005208070), chk1 inhibitors (WO 2006/021002; WO/2006/014359; WO 2006/012308; WO 2005/027907; WO 2002/070494; WO 1999/011795); the contents of the afore-mentioned Patents and Patent Applications are herewith incorporated by reference in their entireties.

Depending on the neoplastic condition, pharmaceutical compositions of the invention can be formulated to include one or more cytokines, lymphokines, growth factors, or other hematopoietic factors which can reduce negative side effects that may arise from, or be associated with, administration of the pharmaceutical composition alone. Cytokines, lymphokines, growth factors, or other hematopoietic factors particularly useful in pharmaceutical compositions of the invention include, but are not limited to, those that are commercially available by such companies as R&D Systems (Minneapolis, Minn.).

When necessary, in order to promote penetration of the blood-brain-barrier (BBB), the active compounds can be administered by using various now strategies for gaining drug access to the brain. Various strategies known in the art for increasing transport across the BBB can be adapted to the compounds of the invention to thereby enhance transport of the modulators across the BBB (for reviews of such strategies, see e.g., Pardridge. Trends in Biotechnol. 12:239-245 (1994); Van Bree, et al. Pharm. World Sci. 15:2-9 (1993); and Pardridge, et al. Pharmacol. Toxicol. 71:3-10 (1992)). In one approach, the compound is chemically modified to form a prodrug with enhanced transmembrane transport. Suitable chemical modifications include covalent linking of a fatty acid to the compound through an amide or ester linkage (see e.g., U.S. Pat. No. 4,933,324 and PCT Publication WO 89/07938; U.S. Pat. No. 5,284,876; Toth, et al. J. Drug Target. 2:217-239 (1994); and Shashoua, et al. J. Med. Chem. 27:659-664 (1984)) and glycating the compound (see e.g., U.S. Pat. No. 5,260,308). Also, N-acylamino acid derivatives may be used in a modulator to form a “lipidic” prodrug (see e.g., U.S. Pat. No. 5,112,863).

In another approach for enhancing transport across the BBB, a peptidic or peptidomimetic compound is conjugated to a second peptide or protein, thereby forming a chimeric protein, wherein the second peptide or protein undergoes absorptive-mediated or receptor-mediated transcytosis through the BBB. Accordingly, by coupling a compound as disclosed herein to this second peptide or protein, the chimeric protein is transported across the BBB. The second peptide or protein can be a ligand for a brain capillary endothelial cell receptor ligand. For example, a preferred ligand is a monoclonal antibody that specifically binds to the transferrin receptor on brain capillary endothelial cells (see e.g., U.S. Pat. Nos. 5,182,107 and 5,154,924 and PCT Publications WO 93/10819 and WO 95/02421). Other suitable peptides or proteins that can mediate transport across the BBB include histones (see e.g., U.S. Pat. No. 4,902,505) and ligands such as biotin, folate, niacin, pantothenic acid, riboflavin, thiamin, pryridoxal and ascorbic acid (see e.g., U.S. Pat. Nos. 5,416,016 and 5,108,921). Additionally, the glucose transporter GLUT-1 has been reported to transport glycopeptides (L-serinyl-β-D-glucoside analogues of [Met5]enkephalin) across the BBB (Polt et al. Proc. Natl. Acad. Sci. USA 91:7114-1778 (1994)). Accordingly, a compound can be coupled to such a glycopeptide to target the modulator to the GLUT-1 glucose transporter. For example, a compound which is modified at a free amine with the modifying group Aic (3-(O-aminoethyl-iso)-cholyl, a derivative of cholic acid having a free amino group) can be coupled to a glycopeptide through the amino group of Aic by standard methods. Chimeric proteins can be formed by recombinant DNA methods (e.g., by formation of a chimeric gene encoding a fusion protein) or by chemical crosslinking of the modulator to the second peptide or protein to form a chimeric protein. Numerous chemical crosslinking agents are known in the art (e.g., commercially available from Pierce, Rockford Ill.). A crosslinking agent can be chosen which allows for high yield coupling of the modulator to the second peptide or protein and for subsequent cleavage of the linker to release bioactive modulator. For example, a biotin-avidin-based linker system may be used.

In yet another approach for enhancing transport across the BBB, the compound is encapsulated in a carrier vector which mediates transport across the BBB. For example, the compound can be encapsulated in a liposome, such as a positively charged unilamellar liposome (see e.g., PCT Publications WO 88/07851 and WO 88/07852) or in polymeric microspheres (see e.g., U.S. Pat. No. 5,413,797; U.S. Pat. No. 5,271,961; and U.S. Pat. No. 5,019,400). Moreover, the carrier vector can be modified to target it for transport across the BBB. For example, the carrier vector (e.g., liposome) can be covalently modified with a molecule which is actively transported across the BBB or with a ligand for brain endothelial cell receptors, such as a monoclonal antibody that specifically binds to transferrin receptors (see e.g., PCT Publications WO 91/04014 and WO 94/02178).

In still another approach to enhancing transport of the modulator across the BBB, the compound can be coadministered with another agent which functions to permeabilize the BBB. Examples of such BBB “permeabilizers” include bradykinin and bradykinin agonists (see e.g., U.S. Pat. No. 5,112,596) and peptidic compounds disclosed in U.S. Pat. No. 5,268,164.

Use of Compounds in the Treatment of Various Disorders

These compound can be used to modulate (i.e., increasing or decreasing) activity of Hsp70 by contacting Hsp70 with a compound as disclosed herein. The modulation can be in vivo, ex vivo, in vitro, or combinations thereof. In jurisdictions where methods of treating a human are barred from being patented, use of a compound in the production of a medicament is contemplated. The use of such medicaments can be for a variety of treatments, including, but not limited to, decreasing aberrant cell proliferation, decreasing beta-amyloid protein aggregation, and/or decreasing polyglutamine protein aggregation. The contacting of the compound of formula (I) can be via administration, admixture, or any other means of allowing interaction between the protein, subject, or sample of interest and the compound of formula (I).

The present invention also includes methods of treating patients suffering from aberrant cell proliferative disorders, such as cancer and tumor therapy or diagnostics. An exemplary method of treatment comprises selecting a patient in need of treatment for a particular proliferative disorder, and administering to the patient an amount of a compound or composition of the invention effective to treat the disorder. The compounds of formula (I) can be used in diagnosing, treating, or ameliorating various cancers or other cell-proliferation disorders, such as basal cell carcinomas, medulloblastoma, gastrointestinal cancers, ovarian fibromas and ovarian dermoids, oral squamous cell carcinoma (OSCC), small-cell lung cancer (SCLC), prostate cancer, rhabdomyosarcomas, malignant gliomas, breast cancer, basal cell carcinoma, medulloblastomas, neuroectodermal tumors, and ependymomas. Selection of the patient involves identifying the proliferative disorder by a review of a patient's medical records, a physical examination, a diagnostic test or interpretation of such test performed on the patient or on a biological sample (tissue, fluid, etc.) from the patient, or the like. Administration of the compound can be by any route of administration, many of which are described herein. The Hsp70 antagonists as disclosed herein can be used to cause transformed cells to become either post-mitotic or apoptotic. Efficacy of treatment is indicated by one or more of the following, for a proliferative disorder: the slowing of cell proliferation, arresting cell proliferation, causing a reduction in proliferated cell mass, eliminating the proliferating cells, reducing or eliminating symptoms associated with cell proliferation, extending life and/or improving the quality of life.

The present invention further provides methods of treating patients suffering from beta-amyloid protein aggregation and/or polyglutamine protein aggregation (such as Alzheimer's Disease and Huntington's Disease). Since Hsp70 can act as a chaperone in protein folding processes, agonists of Hsp70 allow for inhibition or correction of misfolding. Since aggregation of beta-amyloid and polyglutamine proteins is typically due to misfolding of the protein and subsequent association of the misfolded proteins into aggregations and/or fibrils, increased activity of Hsp70 as a chaperone allows for prevention of such misfolding and therefore aggregation, and/or provides a mechanism for refolding of the misfolded proteins. An exemplary method of treatment comprises selecting a patient in need of treatment for a particular protein aggregation disorder, and administering to the patient an amount of a compound or composition of the invention effective to treat the disorder. The compounds of formula (I) can be used in diagnosing, treating, or ameliorating Alzheimer's Disease or Huntington's Disease.

In jurisdictions that forbid the patenting of methods that are practiced on the human body, the following restrictions are intended: (1) the selecting of a human subject shall be construed to be restricted to selecting based on testing of a biological sample that has previously been removed from a human body and/or based on information obtained from a medical history, patient interview, or other activity that is not practiced on the human body; and (2) the administering of a composition to a human subject shall be restricted to prescribing a controlled substance that a human subject will self-administer by any technique (e.g., orally, inhalation, topical application, injection, insertion, etc.); or that a person other than the prescribing authority shall administer to the subject. For each jurisdiction, the broadest reasonable interpretation that is consistent with laws or regulations defining patentable subject matter is intended. In jurisdictions that do not forbid the patenting of methods that are practiced on the human body, the selecting of subjects and the administering of compositions includes both methods practiced on the human body and also the foregoing activities.

The administration of compounds and compositions as disclosed herein can be once daily, twice daily, three times daily, weekly, biweekly, semi-weekly, or monthly. The length of the treatment will typically be as long as desired or as long as safe (e.g., in the absence of adverse side effects or in the absence of severe side effects). Such decisions can be determined by a treating profession in view of a subject's medical history.

For all methods and uses of the invention, co-therapy with two or more compounds of the invention or a second therapeutic agent, simultaneously or in tandem, also is contemplated. In certain cases, the co-therapy sensitizes the effect of the compound of formula (I), the second therapeutic, or both. As used herein, the term “sensitize” means that the effect of the particular therapeutic is enhanced in the presence or co-administration of a second therapeutic.

In a specific embodiment, administration of a compound of formula (I) and a Hsp90 modulator is contemplated. Hsp90 modulators such as those described in U.S. Pat. Nos. 7,160,885; 6,946,456; 6,747,055; and 6,670,348; U.S. Patent Publication No. 2007/0087998; 2007/0072855; 2007/0043044; 2007/0032532; 2007/0027150; 2007/0010432; 2006/0223797; and 2006/0205705; and International Patent Publications WO96/33989; WO98/18780; WO99/55689; and WO02/16369, each of which is incorporated in its entirety by reference herein. Specific Hsp90 modulators include geldanamycin, radicicol, 17-AAG, KOS-953, 17-DMAG, CNF-101, CNF-1010, 17-AAG-nab, NCS-683664, Mycograb, CNF-2024, PU3, PU24FCl, VER49009, IPI-504, SNX-2112 and STA-9090.

Hsp90 is an important cell cycle regulatory protein, implicated in the correct folding of multiple proteins in the mitogenic signal cascade. Hsp90 also plays a role in cyclin dependent progression through G1 and G2 and in centrosome function in mitosis. Hsp90 substrates include a number of steroid hormone receptors including the androgen receptor (AR), estrogen receptor, and glucocorticoid receptor.

Hsp90 has been specifically implicated in the proper folding of a number of tyrosine and threonine kinases. It also insures the correct folding and activity of numerous kinases involved in cell proliferation and differentiation, many of which also play roles in oncogenesis.

Hsp90 also functions as part of a multi-component complex interacting with many other co-chaperone proteins, including Hsp70 (a Hsp70/90 complex). While Hsp90 forms a multi-component complex to some extent in normal cells, nearly all Hsp90 present in cultured tumor cells has been shown to be part of a multi-component complex, e.g., Hsp70/90. A number of known oncogenic proteins that are Hsp90 substrate proteins, depend on the chaperone activity of the Hsp70/90 complex for correct folding. Thus, Hsp90 functions as a supplier of oncogenic proteins in tumor cells. Hsp70/90 complex in tumor cells also exhibits higher ATPase activity than Hsp90 from non-cancerous cell lines.

Geldanamycin, a natural product, is an Hsp90 inhibitor that binds to the ATP binding site of Hsp90 inhibiting ATP hydrolysis but not substrate protein binding. Substrate proteins that reside longer on Hsp90 when ATP hydrolysis is inhibited are ubiquinated, and subsequently degraded. Disrupting the function of the Hsp70/90 complex has been shown to deplete oncogenic kinases (via ubiquitin-mediated proteasomal degradation) and decrease tumor growth. The Hsp70/90 complex present in tumor cells exhibits much higher affinity for geldanamycin and for 17-allylamino-17-demethoxy-geldanamycin (17-AAG), a geldanamycin derivative, than Hsp90 in non-tumor cells. Thus, inhibitors of the Hsp70/90 complex have the ability to convert this protein from a chaperone that insures correct protein folding of oncogenic proteins to a selective protein degradation tool.

Because of its roles in cell cycle control, cell growth, and oncogenesis the Hsp70/90 complex is an important target for anti-cancer therapeutics. The ability of certain Hsp70/90 complex inhibitors to cause this protein complex to selectively target its substrate proteins for degradation makes the Hsp70/90 complex an especially desirable anti-cancer target. Because the Hsp70/90 complex comprises both Hsp 70 and Hsp90, administration of compounds which modulate both Hsp70 and Hsp90 can be beneficial in sensitizing the effect of each in the presence of the other. Thus, in some embodiments, the methods or use of the compounds disclosed herein further comprise administration or use of a second therapeutic, such as a Hsp90 inhibitor, wherein either co- or sequential administration of the second therapeutic sensitizes the effect of the compound of formula (I), or the administration of the compound (I) sensitizes the effect of the second therapeutic.

### EXAMPLES

The following examples are provided to illustrate the invention, but are not intended to limit the scope thereof. Example 1 describes a representative synthesis of compounds of the present invention, which was used to prepare the compounds as shown in Table 1. Example 2 describes the preparation of amyloid-beta. Example 3 describes an MTT cancer cell viability assay. Example 4 describes an amyloid-beta turbidity assay. Example 5 describes the acquisition of amyloid-beta transmission electron microscopy. Example 6 describes an amyloid-beta thioflavin T assay. Example 7 describes aggregation of polyglutamine in yeast in the presence and absence of compounds as described herein.

### Example 1

### Solid Phase Synthesis of Compounds of Formula (I)

Wang resin (100-200 mesh), Fmoc-beta-amino acids and reagents used in peptide synthesis were all purchased from Anaspec Inc. All solvents were purchased from Sigma. Microwave reactions were carried out in a Biotage Initiator EXP. The masses of the purified compounds were confirmed by Micromass LCT Time-of-Flight mass spectrometer with electrospray and APCI. All ¹H NMR spectra were recorded on Varian or Bruker spectrometers (500 MHz) in dDMSO.

As an example of the library synthesis: Fmoc-beta-alanine (10 eq.) was dissolved in dry CH₂Cl₂ and activated with 5 eq. EDC at 0° C. for 30 min. The CH₂Cl₂ was removed under reduced pressure and the activated amino acid was dissolved in DMF. The solution was coupled to Wang resin (swelled in DMF) with of 0.1 eq DMAP. The mixture was stirred at room temperature for 2 h. The coupling efficiency of the first residue (Fmoc-β-Ala) was determined from the equation: Fmoc loading (mmole/g)=A_{290 (sample)}−A_{290 (ref)}/(1.65×mg of resin). The first residue attachment efficiency was estimated by taking out samples of approximately 1-2 mg resin transferring them into 3 ml of 20% piperidine in DMF in two cuvettes. After 2-3 min of stirring, the absorbance at 290 nm was determined, using 20% piperidine in DMF as a reference. The average coupling of Fmoc-β-alanine to Wang resin was 80%. All subsequent amino acid couplings, were performed using microwave irradiation. The reaction conditions (such as pressure, microwave power) were controlled by defining the upper temperature limit. To 1 eq Wang resin, 2 eq of the Fmoc-β-amino acid, 3 eq DIC, and 3 eq HOBt were added. The reaction was performed in DMF (5 ml DMF per 0.25 mmol Wang resin), and the mixture was irradiated with microwaves at 60° C. for 20 min. The coupling yield was estimated using the same method as for the first attachment. The resin was washed with 4×10 mL DMF, followed by incubation with 5 ml 20% acetic anhydride for 20 min in order to block uncoupled sites before Fmoc deprotection. The resin was washed (4×10 mL DMF) followed by deprotection of the N-terminus with 20% piperidine in DMF.

After completion of the peptide portion of the desired compound, 5 eq 1-ureido-butyric acid was coupled to the tripeptide in the presence of 3 eq DIC, and 3 eq HOBt in 10 mL DMF per 0.25 g resin. The reaction was carried out under microwave conditions at 70° C. for 20 min, followed by washing with 4×10 mL DMF. The conditions for the Biginelli reactions were as follows: to 0.250 mmol of resin loaded with 1-ureido-butyric acid (or with tripeptide), 5 ml DMF, 4 eq aldehyde (such as p-bromobenzaldehyde or 2-hydroxy-1-naphthaldehyde), 4 eq keto ester (such as ethylacetoacetate) and 250 μL 4:1 DMF:HCl (conc.) were added. Microwave conditions: 120° C. for 40 min. followed by four washing steps: 3×10 mL DMF, 3×10 ml hexanes, 3×10 mL MeOH, 3×10 mL CH₂Cl₂. The product was cleaved off the resin with 5 mL 1:1 CH₂Cl₂:TFA for 30 min, followed by washing the resin with CH₂Cl₂. The solvent was evaporated under reduced pressure and the product was purified to over 90% using reverse phase HPLC. The HPLC purification steps were carried out on a Beckmann System Gold 128 HPLC on a C18 column (Alltech, Econosil C18, 10μ, length 250 mm, diameter 10 mm). The column was eluted with a gradient of acetonitrile in water (30-80%, gradient: 1% per minute, 0.1% TFA) at a flow rate of 5 ml/min. Peaks were collected and freeze-dried. The lyophilized samples were analyzed by mass spectrometry (all conformed to the expected M+H or M+Na) and select compounds were confirmed by ¹H-NMR.

### Example 2

### Luciferase Refolding Studies

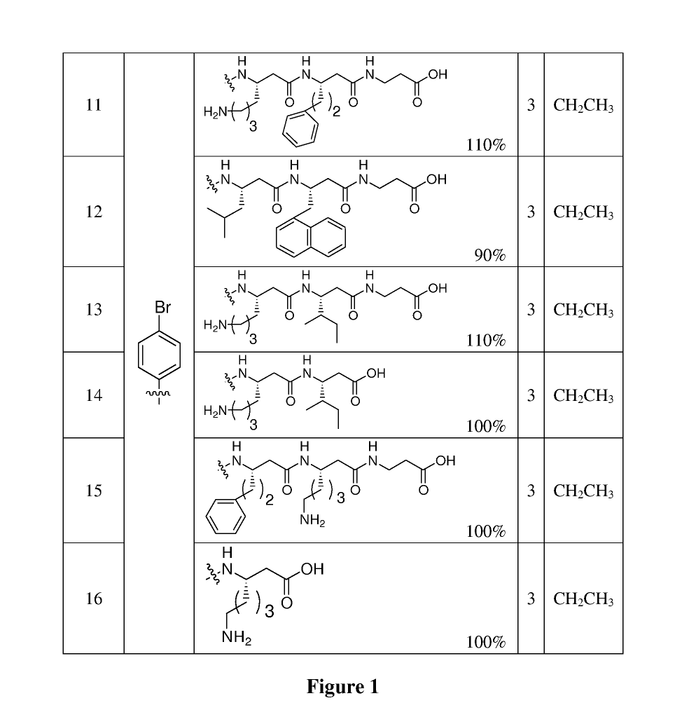

Firefly luciferase (Promega), 0.5 mg/mL, was denatured in Buffer A (25 mM Hepes-KOH, pH 7.2, 50 mM KAc, 5 mM DTT) containing 6 M guanidine hydrochloride (GuHCl) at room temperature for 60 min. The denatured protein was placed on ice for 10 min and diluted 1:40 in Buffer A before refolding. Refolding was initiated by adding 10 mL luciferase into 240 mL refolding buffer (28 mM Hepes-KOH, pH 7.6, 120 mM KAc, 1.2 mM MgAc, 2.2 mM DTT, 1 mM ATP, 8.8 mM creatine phosphate, 35 U/mL creatine kinase, including 15 mL rabbit reticulocyte lysate (RRL) (Promega) and compounds 1-16 (0, 1, 10, 100 mM)). At time intervals of 5 to 10 minutes, 2 mL of the refolding mix was removed and added to 98 mL luciferine solution (0.15 mg/mL luciferine in 25 mM glycylglycine, pH 7.8, 15 mM MgSO4, 5 mM ATP, 2 mM DTT). The progress of refolding was then monitored by immediately measuring luminescence in 96-well, black, flat-bottomed, non-treated, plates (Coming) in a SpectraMax M5 multimode plate reader with 500 ms integration time. FIG. 1 depicts the structures studied and the resulting percent refolding exhibited by luciferase in the presence of the various compounds of formula (I).

### Example 3

### MTT Cancer Cell Viability Assay

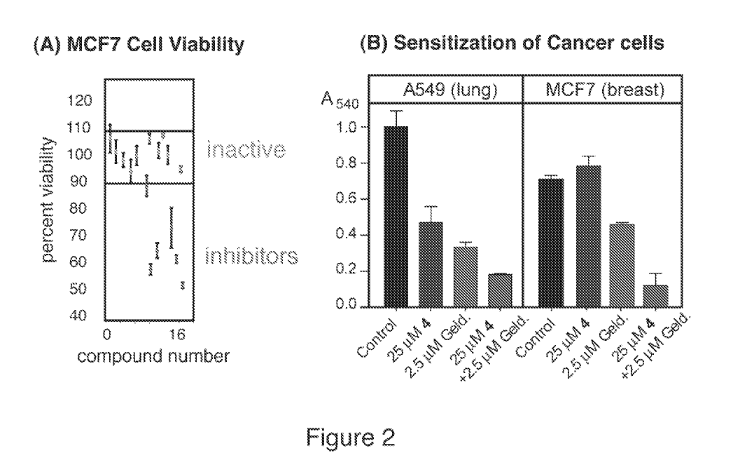

Geldanamycin was obtained from A.G. Scientific, Inc. and the tumor cell lines, A549 (lung) and MCF7 (breast carcinoma), were kind gifts from Dr. Steve Weiss. Cells were maintained in Dulbecco's Modified Eagle's Medium supplemented with 10% fetal bovine serum and antibiotics (100 units/ml penicillin G, 100 μg/ml streptomycin) (Gibco/BRL Life Technologies, Inc., Rockville, Md.) at 37° C. and 5% CO₂. Cells were plated in 96-well plates at 5,000 cells per well. After 24 and 48 h. the cells were treated with compound 4 at concentrations 25, 50 or 100 mM, geldanamycin (2.5, 5 or 10 mM), or a combination of compound 4 and geldanamycin. All compounds were dissolved in DMSO and the final concentration of DMSO after addition of drug was 2%. All experiments were performed in triplicates with DMSO as a reference. On day 3 after the first treatment, the medium was removed and the cells were treated with MTT (3-(4,5-dimethylthiazol-2-yl)-2,5-diphenyltetrazolium bromide, Sigma, St. Louis, Mo.). Briefly, 1 mM MTT was added to the wells followed by incubation at 37° C. for 3 h. The production of blue formazan produced by viable cells was measured in a SpectraMax M5 multimode plate reader at an absorbance of 540 nm. The results are representative of three independent experiments performed in triplicate, error is ±SD. FIG. 2 shows the results of these experiments.

### Example 4

### Amyloid Beta Turbidity Assay

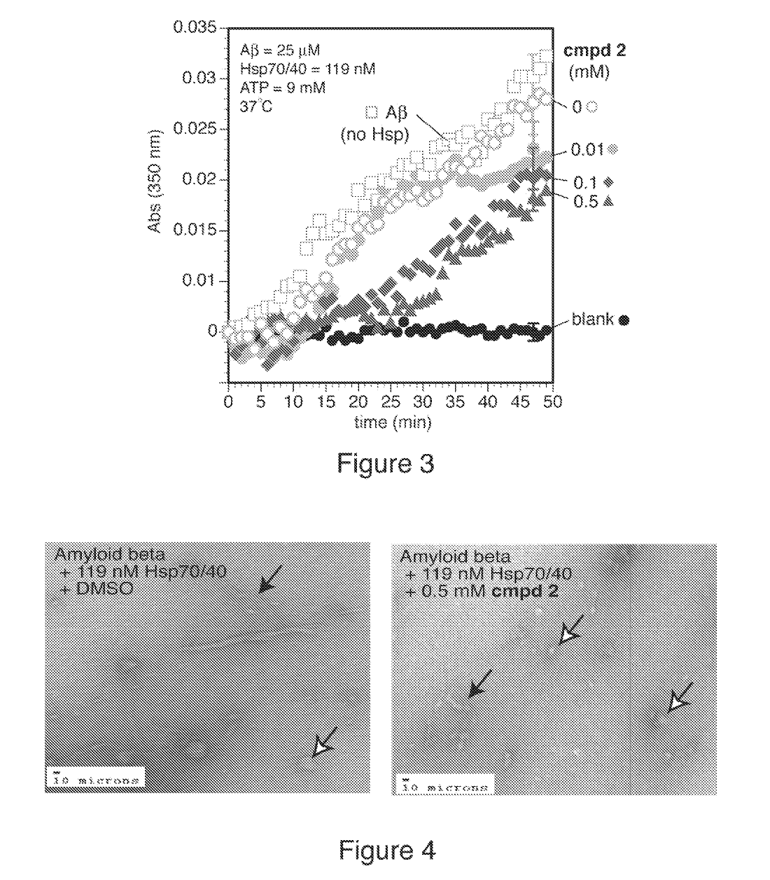

Synthetic amyloid beta 1-42 (AnaSpec, San Jose, Calif.) was prepared for aggregation according to previously developed methods (Fezoui et al., Amyloid 7(3):166-178(2000) and Stine et al, J Biol Chem 278(13):11612-11622 (2003)). Briefly, lyophilized Abeta was resuspended in hexafluoroisopropanol (HFIP), dried under a nitrogen stream, and stored as a film at −20° C. Immediately prior to use, Abeta was resuspended in DMSO to 10 mM and sonicated for 10 minutes. For experiments in which early stages of aggregation were studied, these aliquots were rapidly brought to 25 μM in phosphate buffered saline (PBS) pH 7.2 and used immediately. Human Hsp70, and Hsp40 were provided by Assay Designs (Ann Arbor, Mich.). Concentrated stocks (100×) of these proteins or buffer control were dispensed into the wells of 96-well, half-volume, clear bottom plates (Coming, N.Y.). To these solutions, Abeta in PBS was added to a final volume of 75 μL. Plates were immediately placed in a pre-warmed SpectraMax M5 multimode plate reader and the turbidity measurements initiated. The turbidity program began with a 20 sec mixing shake and was followed by absorbance readings at 330 or 350 nm every 60 sec. A 20 sec shaking step immediately followed each reading, followed by 40 seconds of settling time. The temperature was set at 30 or 37° C. The results of these experiments are shown in FIG. 3.

### Example 5

### Amyloid Beta Transmission Electron Microscopy

At the conclusion of the turbidity measurements, 25 μL aliquots were removed from each well and immediately frozen at −80° C. Thawed samples were placed on glow-discharged Formvar-coated 300-mesh copper grids (Electron Microscopy Sciences) for 1 minute, washed twice with distilled water, and treated with 3% uranyl acetate for 1 minute. Images were taken at 80 kV at magnifications between 46,000× and 130,000×. Image quantification was performed with NIH Image using at least 10 random fields. The microscopy images are seen in FIG. 4.

### Example 6

### Amyloid Beta Thioflavin T Assay

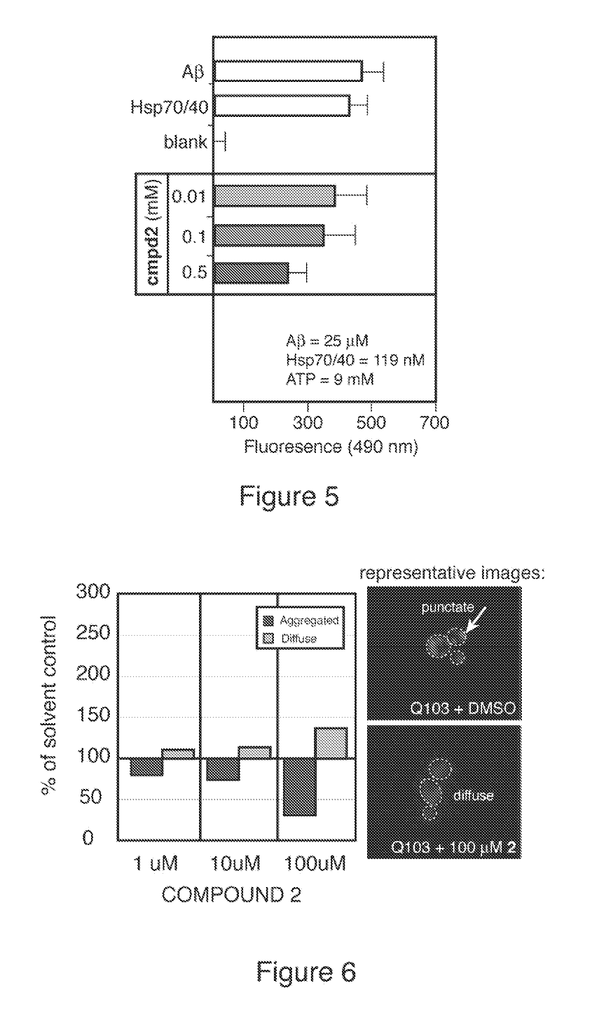

Immediately following removal of samples for electron microscopy, the remaining volume from the turbidity experiments (50 μL) was treated with 75 μL of freshly prepared 50 mM glycine pH 8.0 containing 25 μM thioflavin T. After 10 min at room temperature, the fluorescence was measured on a SpectraMax M5 multimode plate reader using an excitation of 440 nm and emission of 490 nm (475 nm cut-off). The reported values have been corrected by subtracting the background fluorescence of thioflavin T in the absence of amyloid. These results are shown in FIG. 5.

### Example 7

### Polyglutamine Aggregation in Yeast

Sacchromyces cerevesia (Y2269 wt strain) were transformed with a polyglutamine expression construct (pGalQ103Htt), as described in Krobitsch et al., Proc. Natl. Acad. Sci. USA, 97:1589-94 (2000). Two days following transformation, individual colonies were picked and grown in liquid culture (YPAD) for 24 hours at 30° C. Compound 2 was added to these cultures at various concentrations (1 μM, 10 μM, and 100 μM) and incubation continued for another 16 hours at 30° C. A sample of these treated cultures (3-5 μL) were placed on a cover slip and imaged by fluorescence microscopy. Between 200 and 400 individual yeast were scored as either “aggregated” or “diffuse” by hand by at least two separate individuals. All results are reported as a percent of the DMSO control (<1% final concentration). The final results are an average from 3 independent experiments and the images represent typical sub-fields. Between 10 and 50 yeast were typically available in each random field. FIG. 6 shows the results of aggregation and diffusion of yeast in the presence of compound 2.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

What is claimed:

1. A compound having a formula (I): 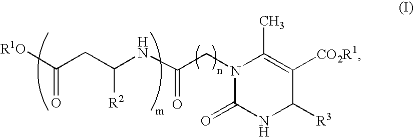
wherein R¹ is independently selected from the group consisting of C₁₋₈ alkyl and H; R² is independently selected from the group consisting of H, C₁₋₈alkyl, C₁₋₈alkylenethiol, C₁₋₈alkylenehydroxy, C₁₋₈alkyleneCO₂H, C₁₋₈alkyleneCO₂C₁₋₈alkyl, C₁₋₈alkyleneC(O)NHC₁₋₈alkyl, aryl, substituted aryl, CH₂aryl, CH₂substituted aryl, CH₂heteroaryl, and CH₂substituted heteroaryl; R³ is selected from the group consisting of aryl, substituted aryl, heteroaryl, and substituted heteroaryl; m is an integer selected from the group consisting of 0, 1, 2, 3, and 4; and n is an integer selected from the group consisting of 1, 2, 3, 4, 5, 6, and 7.

2. The compound of claim 1, wherein R³ is aryl or substituted aryl.

3. The compound of claim 1, wherein R³ is selected from the group consisting of biphenyl, 2-thiophene, 2-hydroxy-1-naphthyl, 4-bromophenyl, 4-nitrophenyl, and 2-chlorophenyl.

4. The compound of claim 1, wherein R¹ is hydrogen, methyl, or ethyl.

5. The compound of claim 1, having a formula selected from the group consisting of:

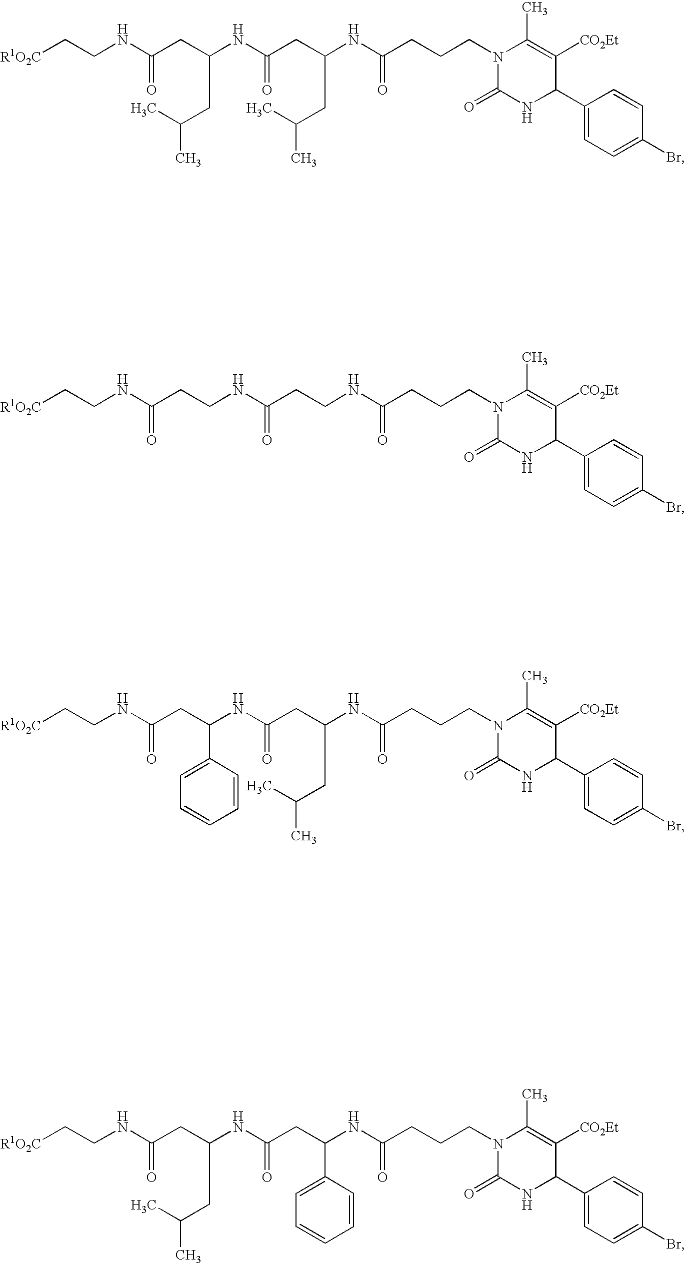

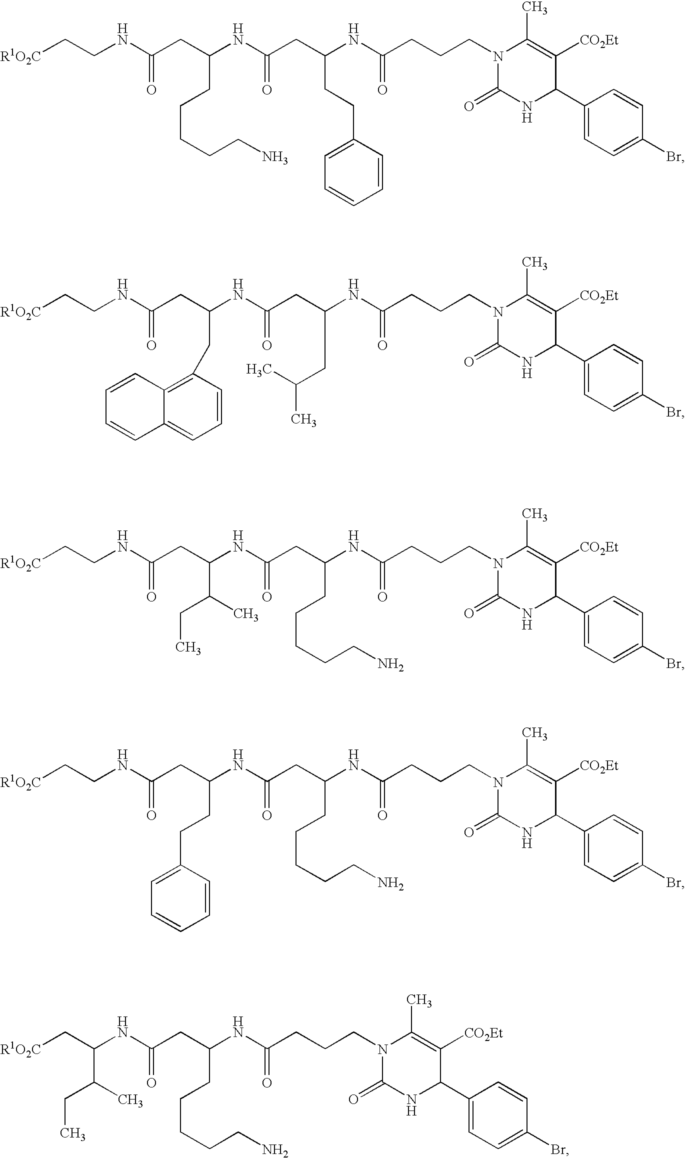

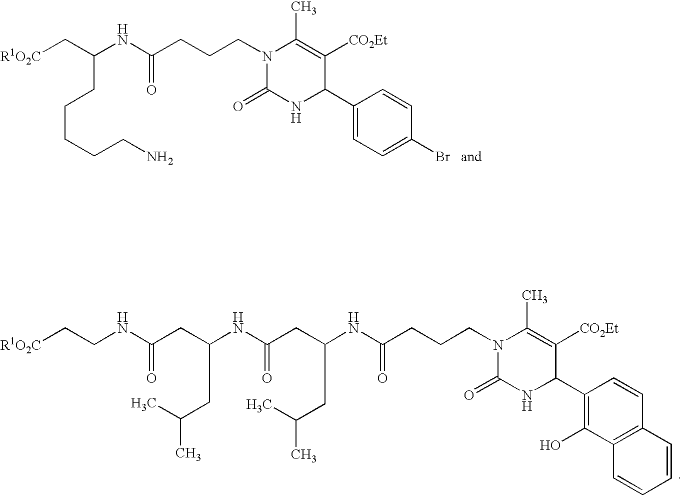

6. A composition comprising the compound of claim 1.

7. A pharmaceutical composition comprising the compound of claim 1.

8. A method of decreasing aggregation of beta-amyloid proteins comprising contacting beta-amyloid proteins with an amount of a compound of claim 1 effective to decrease said aggregation.

9. A method of decreasing aggregation of polyglutamine proteins comprising contacting polyglutamine proteins with an amount of a compound of claim 1 effective to decrease said aggregation.

10. A method of decreasing activity of heat shock protein 70 (HSP70) comprising contacting the HSP70 with an amount of the compound of claim 1 effective to decrease HSP70 activity.

11. A method of decreasing aberrant cell proliferation comprising contacting a cell with an amount of a compound of claim 1 effective to decrease said aberrant cell proliferation.

12. The method of claim 11, further comprising administering a second therapeutic agent, wherein the administration of the compound of claim 1 sensitizes the effect of the second therapeutic agent.

13. The method of claim 12, wherein the compound of claim 1 and the second therapeutic agent are administered simultaneously.

14. The method of claim 12, wherein the compound of claim 1 and the second therapeutic agent are administered sequentially.

15. The method of claim 12, wherein the second therapeutic agent comprises an anti-cancer therapeutic.

16. The method of claim 12, wherein the second therapeutic agent comprises a heat shock protein 90 inhibitor.

17. The method of claim 16, wherein the heat shock protein 90 inhibitor comprises geldanamycin.

18. The method of claim 16, wherein the heat shock protein 90 inhibitor comprises 17-allylgeldanamycin.

19. Use of a compound of claim 1 in the production of a medicament.

20. Use of a compound of claim 1 in the production of a medicament for decreasing aberrant cell proliferation.

21. Use of a compound of claim 1 in the production of a medicament for decreasing beta-amyloid protein aggregation.

22. Use of a compound of claim 1 in the production of a medicament for decreasing polyglutamine protein aggregation.
